(19) United States
(12) Patent Application Publication
(10) Pub. No.: US 2025/0120785 A1
(43) Pub. Date: Apr. 17, 2025

(54) SYSTEMS AND METHODS FOR IMPROVED STEREOTACTIC LOCALIZATION

(71) Applicant: NEXUS SPINE, LLC, Salt Lake City, UT (US)

(72) Inventors: Peter A. Halverson, Salt Lake City, UT (US); David T. Hawkes, Salt Lake City, UT (US)

(21) Appl. No.: 18/917,912

(22) Filed: Oct. 16, 2024

Related U.S. Application Data
(60) Provisional application No. 63/544,582, filed on Oct. 17, 2023.

(51) Int. Cl. A61B 90/10; A61B 90/00

(52) U.S. Cl. CPC A61B 90/10; A61B 90/39

(57) ABSTRACT

Systems and methods for stereotactic localization of components include a system for stereotactic localization of a component during a surgical procedure. In some cases, the system includes one or more of an instrument, a plurality of markers disposed on the instrument, one or more sensors configured to detect a position of each of the markers, and an output device. In some cases, the system is configured to calculate a digital 3-dimensional array based on the position of at least some of the plurality of markers, compute a location of the instrument, and communicate the location of the instrument to a user of the system through the output device. Other implementations are described.

### RELATED APPLICATION

This application claims priority to U.S. Provisional Application Ser. No. 63/544,582 (Attorney Docket No. 23845.160), filed Oct. 17, 2023, and entitled SYSTEMS AND METHODS FOR IMPROVED STEREOTACTIC LOCALIZATION; the contents of which are incorporated herein by reference in their entirety.

### FIELD

The present disclosure relates to surgical procedures, and more particularly to systems and methods for providing improved stereotactic localization of instruments, body parts, and other components during such procedures.

### BACKGROUND AND RELATED ART

Treatment of certain conditions often requires surgical intervention. Surgery generally requires cutting, boring, creating at least one incision, or otherwise opening the skin, organs, or other parts of a patient. Accordingly, while surgery is sometimes necessary, methods for minimizing the damage done to a patient during a surgical procedure are desirable. One issue is that making smaller incisions or otherwise causing less damage to a patient during surgery can make it more difficult for a surgeon to see what is happening inside the patient. Accordingly, certain tools have been developed to aid the surgeon in visualizing the surgery.

One type of tool for visualizing a surgery is stereotactic imaging equipment. In some cases, stereotactic technology can help a surgeon to visualize the positioning of probes or other instruments inside of a patient during surgery by reconstructing digital, 3-dimensional diagrams that represent the positioning of the instruments during surgery.

Unfortunately, many (if not all) existing stereotactic imaging systems have significant issues. For example, some stereotactic imaging systems use one or more metal balls that are configured to be detected by a camera, with such balls being attached to a portion of an instrument exterior to the patient. In many such systems, if one or more of the balls becomes obscured or covered during the operation (e.g., as a surgeon moves between one or more of the balls and a camera, or one ball becomes obstructed by another ball), the stereotactic system fails to display an accurate image (or, in some cases, any image at all). Additionally, the number of localization points can be severely limited by the camera's inability to differentiate between different balls, relying instead on the positions of the balls to determine where the balls are with respect to one another.

Thus, while stereotactic techniques currently exist that are used to identify the location of instruments with respect to a patient, challenges still exist, including those listed above. Accordingly, it would be an improvement in the art to augment or even replace current techniques with other techniques.

### SUMMARY

Oftentimes during a surgical procedure, it is difficult to know where an instrument, an implant, an anchor, or another component is positioned or how it is oriented. This is particularly true where all or part of the component in question is disposed within a patient's body, as is often required during a surgical procedure. To help with this problem, systems can implement stereotactic localization to calculate the position of the component based on available data (e.g., markers that are available for detection, such as by being disposed external to the patient's body), and to display a representation of the position of the component to a user.

Some systems utilize a series of balls disposed on the end of one or more instruments as markers for stereotactic localization, but because the balls are often identical or similar to each other, stereotactic systems can misidentify the balls or confuse them with each other, leading to inaccurate results. Moreover, sometimes one or more of the balls can become obscured (e.g., by a practitioner, a portion of the patient, another instrument, or any other individual or object that may be present in an operating room). When this happens, systems can fail, requiring “blind” operation or pausing the operation to correct the issue so that the stereotactic sensing can resume.

Some implementations of the systems and methods disclosed herein overcome the deficiencies of some previous systems. For example, some implementations include providing a plurality of individually identifiable markers, such as QR codes, AR codes, bar codes, or any other suitable type of individually identifiable markers, which can then be sensed by a detection device and used to computationally form a transformation matrix that can accurately track the position of an object, even where some of the markers are obscured.

Some implementations include a device configured to detect the markers and provide real-time feedback to user. In some cases, the device can display the positions of various components to the user, store positions and other information in memory for later reference, and even calculate and provide recommendations to a user based on the positional data.

Overall, the systems and methods for stereotactic localization disclosed herein provide many advantages over existing systems and methods. The systems and methods include many additional benefits and features which will be readily apparent to the skilled artisan in light of the figures and specification.

### DESCRIPTION OF THE DRAWINGS

The objects and features of the present disclosure will become more fully apparent from the following description and appended claims, taken in conjunction with the accompanying drawings. Understanding that these drawings depict only typical embodiments of the disclosed systems and methods and are, therefore, not to be considered limiting of its scope, the systems and methods will be described and explained with additional specificity and detail through the use of the accompanying drawings in which:

FIG. 1 shows a perspective view of an instrument with a marker guide coupled thereto, in accordance with a representative embodiment;

FIG. 2 shows a perspective view of an instrument with an alternative marker guide coupled thereto, in accordance with a representative embodiment;

FIG. 3 shows a plan view of a calibration guide, in accordance with a representative embodiment;

FIGS. 4A and 4B show digital representations of localized components, in accordance with some representative embodiments;

FIG. 5 shows a perspective view of a detection device, in accordance with a representative embodiment;

FIG. 6A shows a front view of an output and detection device, in accordance with a representative embodiment;

FIG. 6B shows a back view of the output and detection device, in accordance with a representative embodiment;

FIG. 6C shows a partially deconstructed view of the output and detection device, in accordance with a representative embodiment;

FIG. 7 shows a flowchart of a method for localizing an object, in accordance with a representative embodiment;

FIG. 8 shows a diagram of a representative operating environment, in accordance with a representative embodiment; and

FIG. 9 shows a diagram of a network, in accordance with a representative embodiment.

### DETAILED DESCRIPTION

A description of embodiments will now be given with reference to the figures. It is expected that the present systems and methods may take many other forms and shapes, hence the following disclosure is intended to be illustrative and not limiting, and the scope of the disclosure should be determined by reference to the appended claims.

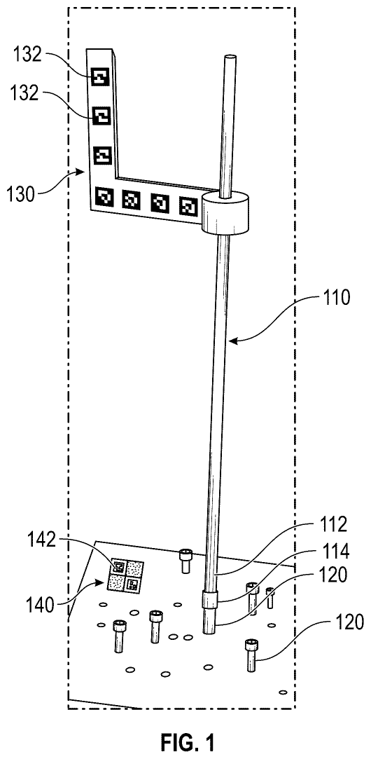

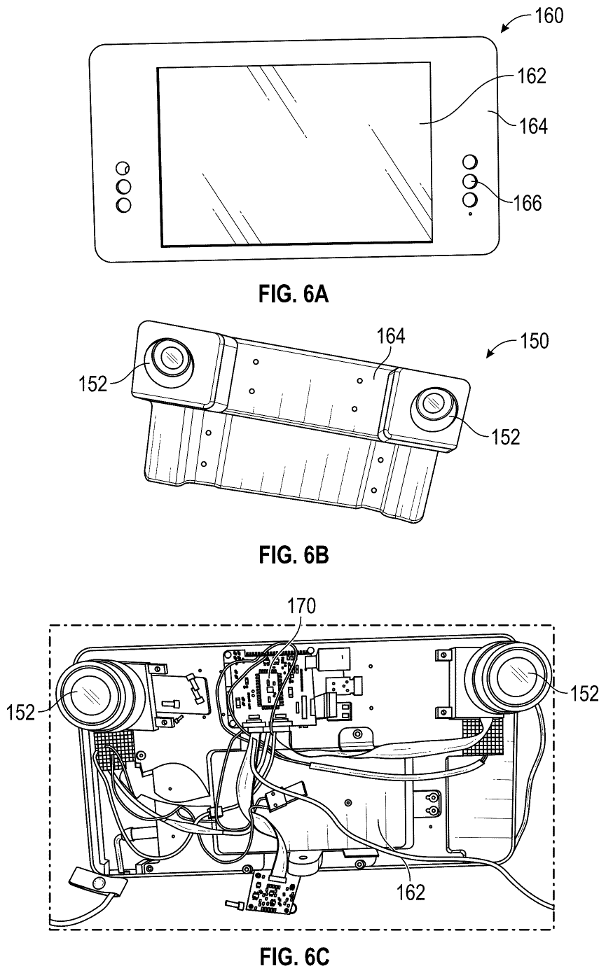

With reference generally to FIGS. 1-6, some embodiments of the described systems and methods include a system 100 for stereotactic localization of one or more components 102 (e.g., an instrument 110, a surgical component 120, a portion of a patient, or any other suitable component (as discussed in more detail below)) during a surgical procedure or any other type of operation, field, process, or procedure, whether related to the medical field or not, in which localization of a component may be useful or desirable (e.g., auto repair, manufacturing, dental care, device repair, bomb diffusion, and any other suitable application). In some embodiments, the described system includes one or more guides, such as a marker guide 130, a reference guide 140, or any other suitable type of guide configured to be detected by one or more detection devices 150. Some embodiments include an output device 160 (e.g., as shown in FIG. 6A) configured to convey information regarding the location of the component in 3D space (e.g., as calculated by a computer 170 based on input from the detection device) to a user. Each of these parts of the system, and additional components that are used as part of the system in some embodiments, are discussed in more detail below.

As mentioned above, in some embodiments, the system 100 is configured to determine the location of one or more components 102 in 3D space. The component can include any part of any object whose precise location in 3D space could be useful to ascertain. For example, some embodiments of the component include one or more of any of the following: an instrument 110, such as one or more probes, tools, surgical instruments, laparoscopic devices, diagnostic devices (e.g., C-arms, probes, thermometers, cameras, sensors, laparoscopic devices, or any other suitable diagnostic devices), or any other instruments of any kind; a surgical component 120, such as an implant or an anchor used in connection with a surgical implement; or a portion of a patient's body (e.g., skin, muscles, tissue, bones, spinal vertebrae, spinal processes, organs, anatomical landmarks, or any other suitable part of the patient). In some cases, the system is configured to stereotactically localize multiple components with respect to each other or with respect to a patient (e.g., by displaying a location of an instrument in relation to a bone, by displaying a bone in relation to another bone, by otherwise displaying multiple components in relation to each other, or by otherwise identifying a location of a component).

As mentioned above, some embodiments of the system 100 include one or more instruments 110. Although the instrument can include any instrument, in some cases, the instrument includes one or more surgical implements, such as screwdrivers, rods, connectors, scalpels, scissors, forceps, clamps, needles, sutures, retractors, suction tubes, suction tips, staplers, cutters, clips, energy systems, laparoscopic instruments, Veress needles, probes, cameras, lenses, light sources, trocars, graspers, tweezers, cauterizers, scopes, catheters, guidewires, stents, implants, consoles, arms, or any other suitable medical devices or apparatuses. By way of non-limiting illustration, FIG. 1 shows an instrument 110 that includes a surgical component configured to install an implant anchor in a patient, in particular being configured to allow a user to position the anchor in the correct location and fasten it in place.

In some embodiments, the instrument 110 includes one or more shafts 112. The shaft can be any part of the instrument configured for holding, connecting multiple parts of the instrument together, providing a frame or body for the instrument, or otherwise imparting the instrument with structure. For example, in some cases, the shaft includes one or more handles, graspers, manipulators, rods, tubes, necks, or other types of instrument shafts). The shaft can be any shape (or have one or more portions that are any straight), such as straight (e.g., forming a cylindrical rod, a square rod, a flat rod, or any other shape extending from one point to another in linear fashion), curved, contoured, bent, wavy, or any other suitable shape. The shaft can also be any suitable length, such as between 1 cm and 1 m (or longer, if needed for operation of a particular instrument, as may be the case with certain catheters or other medical devices) or within any subrange thereof. For example, some embodiments of the shaft are at least 1 cm, 2 cm, 5 cm, 10 cm, 15 cm, or 20 cm long. Some embodiments of the shaft have a length sufficient to allow an operative portion of the instrument (such as a head 114, as discussed in more detail below) to reach an area of interest within a patient. In some embodiments, the shaft has an additional portion of length sufficient to allow the attachment of a marker guide 130 (as discussed in more detail below) to the shaft. Some embodiments of the shaft are rigid (or substantially rigid), while some embodiments of the shaft are flexible (or semi-flexible). By way of non-limiting illustration, FIG. 1 shows an instrument 110 having a substantially straight, substantially rigid shaft with a length sufficient to allow it to be used for installing implant anchors in a patient.

In some embodiments, the instrument 110 includes one or more heads 114. The head can include any portion or portions of the instrument configured to perform a specific technical function, such as cutting, suturing, grasping, coupling, sensing, mating with another component, coupling to another component, or otherwise performing an operational action). For example, some embodiments of the head include one or more bits, tips, blades, needles, graspers, actuators, trocars, sensors, sutures, couplers, jaws, forceps, clips, clamps, bevels, or other functional components. By way of non-limiting illustration, FIG. 1 shows an instrument 110 having a shaft 112 that terminates in a head 114, with the head having a bit configured to rotate an anchor (e.g., a screw) to install the anchor during a surgical procedure. By way of further example, some embodiments of the instrument include a laparoscopy instrument with a shaft (e.g., a laparoscopic needle) and a head selected from any number of clips, applicators, forceps, graspers, trocars, or other heads.

In some embodiments, the instrument 110 is configured to couple to, twist, pull, push, clamp, attach, insert, or otherwise affect or be used in connection with a surgical component 120. While the surgical component can include any tool for use in a medical procedure in connection with the instrument, the surgical component of some embodiments includes one or more implants, anchors (e.g., bolts, screws, staples, or any other suitable type of anchors), tags, bracers, chains, connectors, sponges, or other components that are configured to temporarily, permanently, or semi-permanently be disposed in or on a patient's body. For example, some embodiments of the surgical component include one or more medical implants (such as one or more intervertebral implants, spinal implants, rods, anchors, pacemakers, chips, trackers, monitoring devices, sensors, regulators, stents, or another suitable type of implant). By way of non-limiting illustration, FIG. 1 shows a surgical component 120 in the form of an anchor for use in connection with a spinal implant.

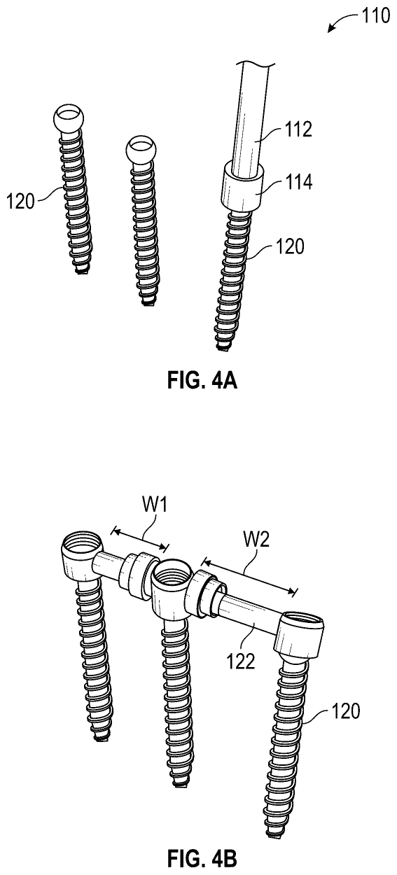

In some embodiments, the surgical component 120 includes or is configured to be used in connection with one or more secondary components 122. In this regard, as with the surgical component, the secondary component can include any surgical component (such as any of the components listed above). By way of non-limiting illustration, FIG. 4B shows a surgical component 120 in the form of an anchor, and a secondary component 122 in the form of a rod, with the system generating a digital representation of the position of the surgical component relative to the secondary component in 3D space.

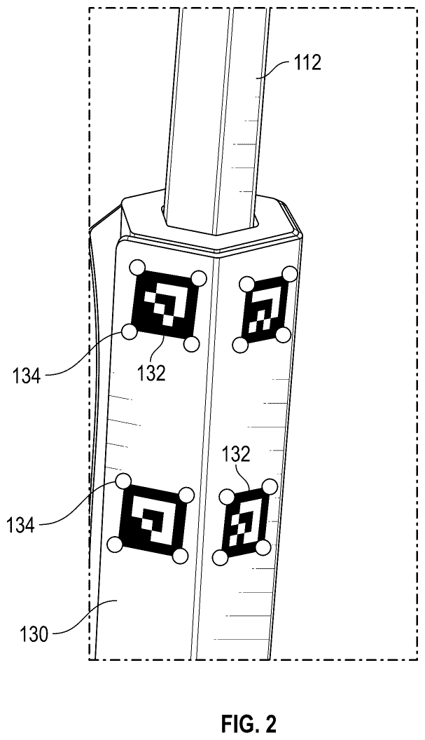

According to some embodiments, the system 100 includes one or more marker guides 130. The marker guide can include anything having one or more markers 132 configured to be detected during a procedure. For example, the marker guide can be a sheet, a strip, a rod, or any other 2D or 3D piece of material capable of bearing one or more markers. In some embodiments, the marker guide includes only a single marker, but in some embodiments, the marker guide includes a plurality of markers. In some embodiments, the marker guide has a specific shape (e.g., circular, semi-circular, triangular, square, rectangular, trapezoidal, pentagonal, hexagonal, star-shaped, T-shaped, L-shaped, polygonal, or any other regular or irregular shape). In some embodiments, the marker guide is sufficiently rigid to ensure that the markers disposed thereon remain fixed relative to each other during a procedure. In some cases, the marker grid is adjustable (in terms of size, shape, weight, location on an instrument 110, or otherwise). By way of non-limiting illustration, FIG. 1 shows an instrument 110 having a marker guide 130 affixed thereto, the marker grid including an L-shaped piece of material having a 2D array of markers 132 disposed thereon, the marker guide extending outwardly from a shaft 112 of the instrument. By way of further non-limiting illustration, FIG. 2 shows a shaft 112 of an instrument having a marker guide 130 wrapped around it.

In some embodiments, the marker guide 130 (or a portion thereof) is disposed on (or is otherwise associated with) the instrument 110. The marker guide can be associated with the instrument in any suitable manner, such as by extending from a portion thereof, being attached to or disposed on a portion thereof, or otherwise being associated with the instrument. In some cases, the marker guide is associated with the instrument in a fixed manner, such that the position of the marker guide relative to the instrument (or a specific portion thereof, such as a shaft 112) is fixed.

In some embodiments, the marker guide 130 is permanently secured to or formed on an instrument 110, a surgical component 120, or another component 102 (e.g., by being printed, engraved, embossed, adhered, molded in, or otherwise permanently or semi-permanently attached thereto). However, in some embodiments, the marker guide provided separately from a component 102 and configured to removably, permanently, or semi-permanently attached thereto. For example, some embodiments of the marker guide are provided as a sticker, a wrap, a stamp, a heat-wrap, a tag, a clip, or another selectively applicable guide. In some embodiments, the marker guide is a single sheet or piece, but in some cases, it is provided as multiple pieces. For example, some embodiments of the marker guide include a sheet configured to be wrapped around the shaft 112 or any other suitable portion of an instrument, and some embodiments of the marker guide include one or more stickers with one or more markers formed thereon, which stickers can then be applied to components as required or desired. By way of non-limiting illustration, FIG. 2 shows a removable marker guide 130 configured to be selectively disposed around an instrument shaft 112.

While the markers 132 can include any indicators capable of being sensed by a detection instrument, in some cases, the markers include one or more visual indicators, such as one or more lines, bars, boxes, spots, colors, patterns, shapes, contours, textures, protrusions, indentations, or any other suitable visual indicators. In some cases, the markers include other types of indicators, such as magnetic, electrical, electromagnetic, sonic, Bluetooth, infrared, ultraviolet, radioactive, radio frequency (e.g., RFID devices), or any other suitable type of indicator capable of assisting a detection instrument in localizing the markers. In some cases, the markers include one or more ArUco (AR) codes, quick response (QR) codes, bar codes, data matrices, PDF417 codes, ARTag codes, AprilTag codes, color codes, or any other type of scannable or visually identifiable code. By way of non-limiting illustration, FIG. 1 shows an instrument 110 with a marker guide 130 affixed thereto, in which the markers 132 comprise AR codes.

In embodiments with multiple markers 132, the markers can be arranged in any suitable manner. For example, in some embodiments, the markers are arranged in one or more: lines, grids, sequences, patterns, or other arrangements. In some embodiments, an arrangement of markers includes at least a 1D array, and in some cases, at least a 2D array, and in some cases, a 3D array. In some embodiments, an arrangement of the markers is regular, while in others the arrangement is irregular. In some cases, it is symmetrical, while in others, it is asymmetrical. In some cases, two or more markers are aligned with each other, while in other cases, two or more markers are offset from one other. In some embodiments, two or more markers are configured to face a same direction, while in some embodiments, two or more markers are configured to face different directions. In some embodiments, markers are each disposed opposite to each other (e.g., to help identify a rotation of a device). In some cases, markers are disposed around a circumference (or any other suitable measurement or location) of an instrument (or part thereof) such that at least one marker is almost always, if not always, visible from any angle at which the instrument may be disposed. Stated another way, in some embodiments, the markers 132 are disposed around the shaft 112 of the instrument 110, such that no matter the orientation of the shaft 112, at least some of the markers 132 are visible to the sensors 152. In some embodiments, the markers 132 are disposed such that the markers 132 are disposed in multiple different planes or otherwise at multiple different angles. In some cases, one or more markers is positioned substantially perpendicular to one or more other markers, and in some cases, at least some of the markers are disposed at a different angle to one or more other markers in multiple dimensions (e.g., one marker is substantially perpendicular to another marker along the x-axis, while another marker is substantially perpendicular to the first marker along the y-axis). By way of non-limiting illustration, FIG. 1 shows a marker guide 130 with a 2D array of markers 132 forming an L-shape, with each marker facing the same direction. By way of further non-limiting illustration, FIG. 2 shows a shaft 112 of an instrument having a marker guide 130 wrapped around it, with markers 132 disposed in a 3D array around the marker guide, with some of the markers facing in one direction and other markers facing in another direction, such that at least one marker is entirely visible from any angle (e.g., the entirety of at least one marker can be seen no matter to which degree the shaft is rotated).

In some embodiments, two or more of the markers 132 are substantially identical to each other, but in some embodiments, some or all of the markers are unique and individually identifiable. As an example, in some embodiments, each of the markers includes a code or other identifier (such as an AR code) that is unique from each other code or identifier. Accordingly, in some embodiments, each of the markers can be readily individually identified without respect to information external to the marker (such as the marker's surroundings, position in relation to other markers, previous locations, or other external information). In some embodiments, the codes have different shapes or configurations (e.g., different combinations of white and black squares disposed throughout the code, or any other suitable combinations of shapes, colors, lines, characters, or other configurations). In some embodiments, the codes incorporate colors to differentiate them from each other.

In some embodiments, one or more of the markers 132 includes one or more individually identifiable points, such as identification points 134, or other identifiers. For example, in some cases, the marker includes a code (e.g., an AR code) having multiple individually identifiable points, such as at each of the corners of the code (e.g., the four corners, where the code is substantially square or rectangular, or at one or more other corners (i.e., all) where the code has a different shape). Thus, in some cases, each marker can contribute multiple location points for use in assembling a code array or matrix. Additionally, in some cases, the multiple individually identifiable points of a marker enable a detection device to more easily identify a rotation of the marker. By way of non-limiting illustration, FIG. 2 shows a plurality of markers 132 in the shape of polygons (e.g., rectangles or squares) having identification points 134 at the corners (note that FIG. 2 shows circles at the identification points 134, but identification points can include any feature, including ordinary corners, particular portions of codes, geometric shapes, or any other feature).

In some embodiments, there are at least one, two, three, four, five, six, seven, eight, nine, ten, or any other number of markers 132 included. Indeed, in some cases, there are at least three markers.

The system 100 can include any number of marker guides 130. In this regard, different marker guides can be associated with different instruments 110, components 102, patients, or other objects or aspects of the system. For example, some embodiments include a specialized marker guide or reference guide 140 including one or more reference markers 142. In some cases, the reference markers are disposed on or coupled with one or more portions of the patient's body. For example, in some cases, one or more reference markers are disposed on a spinal process, and one or more references markers are disposed on an instrument. In some cases, the system is configured to determine a position of a marker 132 in relation to a reference marker 142 in order to calculate or otherwise determine a position of the instrument 110 in relation to a portion of the patient's body. By way of non-limiting illustration, FIG. 1 shows a reference guide 140 containing a plurality of reference markers 142 disposed on an object configured to receive a surgical component 120.

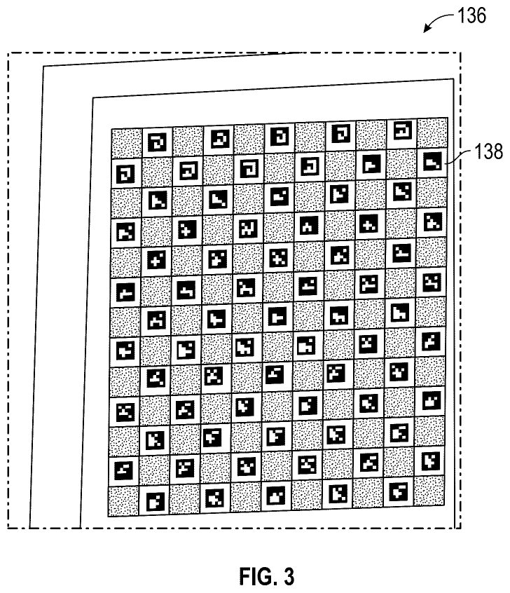

Some embodiments include one or more calibration guides 136 for use in calibrating the system 100. The calibration guide can include anything configured to calibrate hardware that can be used in connection with the system. Such calibration can include any suitable calibration. By way of non-limiting illustration, FIG. 3 shows a calibration guide 136 having a plurality of markers 132, which can be used to calibrate equipment associated with the systems and methods disclosed herein.

According to some embodiments, the system 100 includes one or more detection devices 150. The detection device can include any component configured to detect a position of a marker 132 (or reference marker 142) or to aid in such detection. In some embodiments, the position of the marker to be detected includes one or more of an x-position, a y-position, a z-position, an x-rotation, a y-rotation, and a z-rotation. Indeed, in some embodiments, the position includes two or more of the x-position, the y-position, the z-position, the x-rotation, the y-rotation, and the z-rotation. In some embodiments, the position includes three or more of the foregoing, four or more of the foregoing, five or more of the foregoing, or each of the x-position, the y-position, the z-position, the x-rotation, the y-rotation, and the z-rotation. For clarity, where a marker is substantially in the form of a 2D square, the position can include rotation in its same 2D plane (e.g., rotation along the z-axis, or z-rotation), or a pitched or angled rotation along the x-axis or the y-axis (x-rotation or y-rotation), thereby shifting the 2D square into a new 2D plane. Regarding position, a 2D marker can move along the x- or y-axis to a new position in the same 2D plane, or it can move along the z-axis to a new position in a new plane.

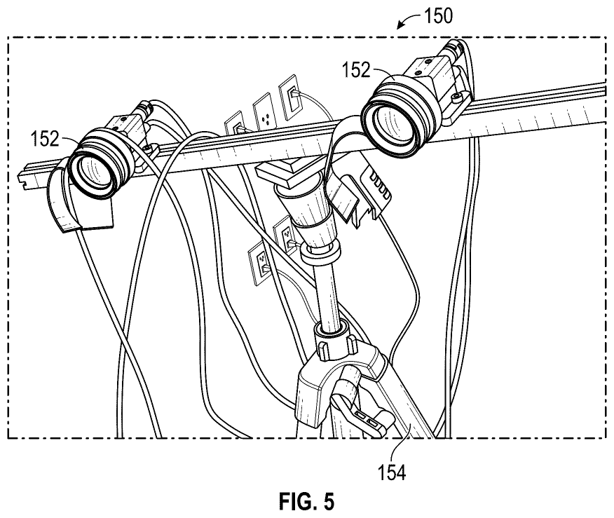

In some embodiments, the detection device 150 includes one or more sensors 152. The sensor can include any component configured to sense the position of a marker 132 in any manner, including one or more film cameras, digital cameras, optic cameras, infrared cameras, microphones, Geiger counters, magnetic detectors, proximity sensors, accelerometers, thermometers, pressure sensors, position sensors, motion detectors, photodetectors, photoelectric sensors, RFID scanners, radar detectors, transmitters, receivers, scanners, photodiodes, charge-coupled devices, ultrasound sensors, or any other type of sensors capable of detecting a marker 132. While some embodiments include only a single sensor, some embodiments include two or more sensors. In some embodiments, at least two sensors 152 are spaced apart by a distance, providing a binocular or stereoscopic sensory system. In some cases, the distance is between approximately 0.5 cm and 2 m, or any subrange thereof. For example, in some embodiments, the distance between a center of two sensors is approximately 30 cm±10 cm. In some embodiments, three or more sensors are used. In some such cases, the sensors can be separated by any suitable distance, and arranged in any suitable manner in 2D or 3D space. In some embodiments, the sensors are set at different angles or in different positions around the operation area such that they may sense markers from a variety of different angles or positions. By way of non-limiting illustrations, FIGS. 5 and 6B-C show detection devices 150 having two digital optic sensors (in the form of video cameras) spaced apart by a distance to allow for binocular sensing of markers.

In some embodiments, the detection device 150 is configured to be mounted in a position where the sensors 152 can perceive one or more of the markers 132. Indeed, the detection device can have any feature allowing it to be used to detect the markers. In some embodiments, the detection device is configured to be maneuverable into a variety of positions to receive a better view of one or more markers. In some embodiments, the detection device is configured to be selectively held by an operator and easily maneuverable. For example, in some embodiments, the detection device is relatively light (e.g., between 1 g and 10 kg, or any subrange thereof, such as approximately 1 kg). In some cases, the detection device 150 is relatively small, such as approximately the size of a digital tablet device (e.g., having a length between 10 cm and 75 cm, a width between 5 cm and 50 cm, and a thickness between 1 mm and 10 cm, including any subranges of any of the foregoing). In some embodiments, the sensors 152 are disposed on opposite sides of a same face of the detection device 150. In some embodiments, the detection device 150 includes a stand 154, such as a mount, a tripod, or another kind of stand. In some cases, the stand is adjustable, allowing the sensors 152 to be (individually or collectively) placed into desired positions. In some embodiments, the sensors 152 are individually adjustable in any manner in which sensors can be adjusted, and in some embodiments, the sensors 152 are collectively adjustable in any manner in which sensors can be adjusted. Examples of suitable adjustments for sensors include focus, resolution, focal length, sensory target, wavelength, frequency, sensitivity, polarity, mode, range, limits, or other similar adjustments.

According to some embodiments, the system 100 (or a part thereof, such as the detection device 150) includes or is otherwise associated with an output device 160. The output device can include any component useful for communicating information (such as the location and position of markers 132, the instrument 110, the surgical components 120, portions of a patient, or any other information that may be valuable during a medical procedure) to a user of the system. In some embodiments, the output device is configured to communicate information visually, such as via a screen 162, a projector, monitor, an augmented reality display, video display glasses, or any other suitable visual display. In some embodiments, the output device 160 is configured to communicate information through sound, tactile feedback, vibrations, or in any other manner (instead of or in addition to visual communication). By way of non-limiting illustration, FIG. 6A shows an output device 160 having a screen 162 configured to show the positions of one or more components used in a procedure.

In some embodiments, the output device 160 is coupled with the detection device 150. In such cases, the output device can be coupled with the detection device in any suitable manner, such as physically, functionally, communicatively, wirelessly, via a wired connection, or in any other suitable manner. In some embodiments, the output device is in communicative connection with the detection device, thereby allowing the output device and the detection device to exchange signals. In some embodiments, the output device is integrally formed with the detection device into a single unit. By way of non-limiting illustration, FIGS. 6A-6C show an integrated device combining the capabilities of a detection device 150 (with sensors 152 for detection) and an output device 160 (with a screen 162 for providing output).

According to some embodiments, the system 100 includes one or more processors, CPUs, or other computer components configured to operate as a computer 170 (as discussed in more detail below in the REPRESENTATIVE OPERATING ENVIRONMENT Section of this disclosure). In some embodiments, the computer is configured to calculate a digital 3-dimensional array based on the position of at least some of the plurality of markers 132 or reference markers 142. In some embodiments, the computer is configured to compute a location or position of the instrument 110, and to communicate the location or position of the instrument to a user of the system through the output device 160. Accordingly, the computer can include any component for computing and rendering a stereotactic representation of one or more components 102 or any related information.

In some embodiments, the computer 170 is configured to calculate, render, generate, or otherwise create information configured to be communicated to a user. For example, in some cases, the output device 160 has a digital display configured to show a 3D representation of one or more components 102 (e.g., instruments 110, surgical components 120, body parts of a patient, or any other suitable objects). In some cases, the digital display is configured to represent various objects in different colors. In some cases, the digital display is configured to generate a high-contrast image so that the representation of objects in 3D space is easy to perceive and understand.

In some embodiments, the computer 170 is configured to provide feedback relating to the location of objects or other aspects of the procedure (e.g., using the output device 160). For example, in some cases, the computer (together with the output device) is configured to provide feedback in which one or more of the following occurs: a digital representation of one or more components changes color; the device vibrates or otherwise provides tactile or sensory feedback; a sound is played (e.g., a chime, an alarm, a verbal communication, or any other suitable sound is produced); a digital representation of a component changes shape or size; a digital representation of a component wiggles; a digital representation of a component is outlined; a message is displayed; a warning icon is displayed; numbers or calculations are displayed; an overlay of an ideal position is displayed next to a rendering of the current position of a component; or any other suitable type of indicator is displayed or produced. The foregoing feedback can be provided in response to a broad range of occurrences, such as if, according to the computer's calculations: a component is inserted too deep; a component is inserted at an incorrect angle; the structural integrity of a component is compromised; there is a computational error; the computer is unable to accurately detect the markers or compute the location of a component; there is a hardware error; there is additional helpful information to be provided to a user (e.g., a correct location for placement of an implant; a correct size of a component); a user-specified stimulus occurs; a specific time interval elapses; a patient metric is triggered (e.g., a change in vital signs); a proper (or improper) component location is achieved; a calculation is completed; or any other trigger event occurs.

Although any suitable portion of the instrument 110, the surgical component 120, the patient, and any other suitable component can be flexible or resilient, in some embodiments, at least one of the instrument 110, the surgical component 120, a portion of the patient, and another component 102 includes one or more rigid bodies. A rigid body is an object that generally substantially retains its shape. As examples, a metal rod that is substantially rigid, a bone, a screw, a brick, and other substantially rigid components each qualify as rigid bodies. In this regard, because rigid bodies substantially retain their shape, a position of one part of a rigid body can be calculated based on a position of another part of the rigid body. To illustrate, the position, location, and orientation of one end of a metal rod can be calculated based on the position, location, and orientation of the other end of the metal rod, because the two ends are locked in a positional relationship determined by the configuration of the rigid body. In some embodiments, one or more markers 132 are placed on one or more parts of a rigid body (e.g., the shaft 112 of the instrument 110, a spinal vertebra of a patient, or another rigid body), and the computer 170 is configured to calculate the position and orientation of the entire rigid body based on the position and orientation of the markers and one or more known dimensions of the rigid body. In some cases, markers are placed on multiple rigid bodies, and the computer is configured to calculate the position and orientation of each of the rigid bodies (individually or in relation to each other) based on the positions and orientations of the markers. In some embodiments, the computer is calibrated or the specifications of the rigid body are provided to the computer (e.g., through scanning, manual entry, or any other manner) to enable to the computer to calculate the position and orientation of the full rigid body based on a sensed position and orientation of part of the rigid body.

In some embodiments, the computer 170 is configured to combine one or more (e.g., all) the markers 132 (or one or more of the identification points 134 of each of the markers 132) detected by the sensors 152 into an array and map the rigid body in relation to the array. In some cases, even where one or more markers 132 (or identification points 134 thereof) are obscured (e.g., by part of the instrument 110, by a practitioner or user of the system, by a part of the patient's body, or otherwise), the computer 170 calculates the array based on the sensed markers 132 and fills in the missing information by extrapolating from the sensed data. In some embodiments, the computer 170, calculates the array based on only the sensed markers or identification points (in some embodiments, this includes the markers or identification points sensed in a single image, and in some embodiments, this includes the markers or identification points sensed across multiple images (or the markers or identification points sensed across multiple or all images)) and excludes from analysis the markers or identification points not directly sensed. In some embodiments, the computer is configured to take into account sensed markers or identification points as well as calculated (e.g., extrapolated) markers or identification points. That said, in some embodiments, the computer assigns a weight to makers or identification points, and in some cases the computer gives a greater weight to sensed markers or identification points than to extrapolated markers or identification points. In some cases, markers or identification points with a greater weight preferentially influence the computer's calculations (e.g., in computing the position of a rigid object, markers or identification points with a greater weight can influence the calculations more heavily than markers or identification points with less weight). Further, in some embodiments, by using a plurality of markers or identification points formed into an array (in some cases, with the various markers or identification points being weighted), a margin of error for a calculated position and orientation of a rigid body is decreased. By way of example, a first marker may appear in 10 images, and a second marker may appear in 20 images, and the computer may assign a greater weight for calculations to the second marker.

In some embodiments, one rigid body bearing one or more markers 132 is configured to couple to another rigid body not having markers 132. For example, in some cases, the head 114 of an instrument 110 bearing markers 132 on its shaft 112 is configured to couple to a surgical component 120 (such as a screw or another anchor or an implant body) or any other suitable component 102, which in some cases does not directly bear a marker 132. In some such cases, the interaction between the first rigid body (e.g., the instrument 110) and the second rigid body (e.g., the surgical component 120) is known (e.g., calibrated, sensed, calculated, entered, or otherwise known), such that the computer 170 can calculate the position and orientation of the second rigid body based on the sensed and calculated position and orientation of the first rigid body (e.g., based on the digital 3-dimensional array constructed from the sensed markers 132). In some embodiments, the computer 170 is configured to remember (e.g., store in its memory) the position, size, and orientation of the second rigid body, and communicate such position and orientation (e.g., through the output device 160) to a user of the system, in some cases even after the first rigid body is no longer coupled with the second rigid body. For example, in some embodiments, the computer calculates the position and orientation of an instrument and a surgical component configured to be implanted into a patient using the instrument, and after the surgical component is implanted (e.g., released by the instrument and embedded in the patient), the computer continues to communicate the position and orientation of the implanted surgical component to the user.

In some embodiments, the system 100 (e.g., via the computer 170) is configured to calculate a configuration for one or more secondary components 122 based on stored information regarding the position and orientation of one or more surgical components 120. For example, in some embodiments, one or more surgical components, such as anchors (e.g., screws), are embedded into adjacent spinal vertebrae (in some cases, using the instrument 110). In some cases, in order to lock the surgical components into position, a secondary component (such as an implant body, a rod, or any other suitable component) needs to be affixed to the surgical components. Some operations use a pre-formed rod with a size and shape based on pre-operative measurements. Such a pre-formed rod, however, may not be an exact fit for surgical components, as the surgery can change the actual and desired configurations. In contrast, in some embodiments of the present system, the computer is configured to calculate an optimal configuration for the secondary component based on the actual positions and orientations of the surgical components or markers 132 or reference markers 142, as measured and stored during the surgical procedure. In some embodiments, the secondary component 122 is formed (e.g., it is created to specific parameters, such as size, shape, length, rigidity, material, angle, composition, or other characteristics) based on the calculated optimal configuration. By way of non-limiting illustration, FIGS. 4A-4B show a digital representation (as may be rendered by a computer and shown on a digital display) of surgical components 120 as installed by an instrument 110. The position of the instrument 110, and congruently, the surgical components 120 (in this case, implant anchors), is calculated by the computer based on the positions of the markers (not shown). The positions of the surgical components 120, once uncoupled from the instrument 110 (e.g., by being implanted in a bone of the patient), are remembered by the computer and continue to be rendered and displayed to the user. Then, as shown in FIG. 4B, the proper dimensions of a secondary surgical component 122 (in this case, an implant body configured to join each of the anchors together) can be calculated and displayed to a user.

To expound on the foregoing paragraph, some embodiments of the secondary component 122 are formed from a plurality of interchangeable segments. In some embodiments, the computer 170 further calculates whether installation of a secondary component 122 is feasible, and provides a warning or guidance for adjusting the surgical component 120 if the surgical component is not installed in a way that would allow for a desired connection with a secondary component. For instance, some embodiments indicate when an implant has been inserted too far, not far enough, or to a proper depth in a patient.

In some embodiments, reference markers 142 are placed on one or more spinal vertebrae (or other portions of a patient), and at least some rigid body positions and orientations are calculated with respect to such reference markers 142. In some embodiments, reference markers 142 are placed on one or more vertebrae (or other rigid bodies of a patient), and the positions and orientations of the vertebrae (or other rigid bodies) with respect to each other are also calculated. One advantage of the foregoing is that while some systems treat the spine of a patient as a single rigid body, by treating each vertebra or multiple vertebra as different rigid bodies that can move with respect to each other, a greater degree of accuracy can be achieved, especially since vertebrae can shift during an operation. While such markers can once again include any suitable markers, in some cases 2D markers can be particularly useful for use as reference markers, as they can provide the identification points while taking up less room than some 3D markers, thereby avoiding interfering with an operation. Additionally, such markers can be easily applied (e.g., by disposing a sticker, a clip, or another selectively disposable marker on an appropriate reference point). Indeed, in some cases, markers are printed or inked onto desirable reference points.

According to some embodiments, a kit for stereotactically localizing one or more items during a surgical procedure is provided. While the kit can include any component useful for forming, utilizing, or modifying the system, some embodiments of the kit include one or more instruments 110, surgical components 120, secondary components 122, marker guides 130 (including one or more markers 132), reference guides 140 (including one or more reference markers 142), detection devices 150 (e.g., having one or more sensors 152), output devices 160 (e.g., having one or more screens 162), and computers 170 (e.g., specialized to perform the functions discussed herein).

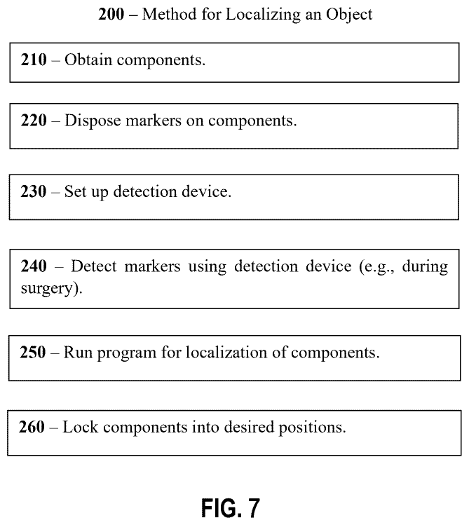

While the described system 100 can be used in any suitable manner, FIG. 7 shows a flowchart illustrating portions of a method 200 of localizing a component (e.g., during a surgical procedure). One or more portions of the described method can be reorganized, omitted, substituted, performed in parallel, performed in series, performed in part, augmented, added to, or otherwise modified in any suitable manner.

As shown in FIG. 7 at box 210, some embodiments of the method 200 include obtaining one or more of the components described herein (e.g., any component making up a part of the system 100). In some such cases, obtaining components includes forming, manufacturing, modifying, using, purchasing, renting, or otherwise obtaining any component discussed herein in any reasonable manner as would be understood by the person of ordinary skill in the art. In some cases, this includes generating one or more codes (e.g., AR codes) for use as markers 132. In some cases, the codes are randomly generated, and in some cases, the codes are procedurally generated (e.g., to ensure that each of the codes is unique or otherwise distinguishable from each of the other codes).

Box 220 illustrates that some embodiments include disposing one or more markers 132 (e.g., as part of one or more marker guides 130) on one or more components 102 (for example, on one or more instruments 112, on one or more surgical components 120, on one or more portions of a patient, or on any other component of the system 100). In this regard, the markers can be disposed on the instrument in any reasonable manner, such as by sticking (or otherwise adhering), wrapping, pinning, clamping, welding, soldering, engraving, embossing, inking, printing, painting, drawing, clipping, screwing, bolting, stapling, etching, molding, attaching with magnets, or otherwise coupling the markers to the component in question. In some embodiments, the markers are disposed on the component in a single dimension (e.g., in a line along a portion of the instrument). In some embodiments, the markers are disposed on the component in at least two dimensions (e.g., forming a 2D grid, array, pattern, or other arrangement). In some embodiments, the markers are disposed on the component in three dimensions (e.g., occupying multiple positions in 3D space, or being offset along each of an x-axis, a y-axis, and a z-axis, or having various rotations or orientations along one or more such axes). In some cases, the markers surround a portion of the component, such as by being wrapped around a shaft of an instrument.

In some embodiments, the method 200 includes disposing reference markers 142 on one or more portions of the patient (e.g., on spinal vertebrae or in any other suitable location). Reference markers can be disposed on a patient (or another frame of reference) in any suitable manner, including via one or more of the coupling techniques discussed in the previous paragraph. Additional examples of potential reference points for reference markers include a bone, a ligament, a muscle, a tissue, an existing implant, skin, a surface external to the patient (such as a table, a strap, or another external object), or another portion of (or adjacent to) the patient. That said, some portions of a patient tend to be flexible and can shift during surgery (e.g., muscles, skin, soft tissues, etc.). Accordingly, some embodiments expressly include disposing one or more reference markers on one or more rigid bodies, such as bones, spinal segments, and certain existing implants, to ensure that the reference markers do not shift relative to other portions of the patient pertinent to the procedure. In some cases, vertebrae can shift with respect to each other, so some embodiments involve placing reference markers on each vertebra involved in the procedure.

With reference now to box 230, some embodiments of the method 200 include setting up a detection device. In some embodiments, setting up the detection device includes physically setting up of one or more sensors, such as cameras (e.g., placing the cameras in a desired location with respect to a surgical field). In some cases, the sensors are spaced a distance apart to provide a binocular or stereoscopic view. In some cases, the orientation of the sensors is fixed to provide a maximum view at a desired distance. In some embodiments, the method 200 includes fixing an aperture of each camera, focusing each camera, or otherwise optimizing camera settings. In some embodiments, setting up the detection device includes defining a relative position (e.g., location and orientation) of the detection device (or part thereof, such as one or more sensors) with respect to a surgical field. In some cases, the relative position of the cameras is known (e.g., measured, set to equal, or otherwise ascertained) beforehand. In other cases, the relative position of the cameras is calculated or computed (e.g., using an object or array of a known size or calibration markers 138, the computer can determine how far the object or array is positioned from the sensors based on sensed input (e.g., perceived size)).

In some embodiments, the method 200 includes defining one or more of the markers. For example, some embodiments include defining the X, Y, Z, or other coordinates of marker locations as they relate to one or more rigid bodies (e.g., the instrument or another component). Some embodiments include defining the markers in relation to a rigid body origin (such as an stl. or obj. origin or another origin as calculated or integrated in a digital file representing the rigid body). In some embodiments, defining a marker is done via manual or automatic inputs in computer software (in some cases, during the manufacturing process or before the system is provided to practitioners for use, but in some cases, this is done around the time of the procedure, or at any other suitable time). In some embodiments, the computer software is configured to create a 3D digital marker based on the markers, and to map the digital marker to a digital representation of a rigid body. In some embodiments, the method includes defining one or more rigid bodies in connection with defining the markers (e.g., by entering the dimensions of the rigid body, scanning the rigid body, selecting the rigid body from a collection of pre-programmed digital representations, or by otherwise defining the rigid body in any other suitable manner). By way of non-limiting illustration, the computer software may include an automated definition module or function (such as an NSMarker_Helpers.createboard function) configured to create a 3D polygonal marker structure based on a variety of factors such as a prescribed normal, marker IDs, size (e.g., of the rigid body), and any other relevant factors. Thus, the 3D polygonal marker can provide a digital representation of the relevant rigid body. By way of further illustration, the computer software can include a manual entry module or function (such as a manualmarker.py function) configured to allow input of x, y, and z coordinates (and in some cases, rotations as well) by hand.

Some embodiments of setting up the detection device include calibrating the detection device. Calibrating the detection device can be done in any suitable manner, such as by running one or more modules or functions, comparing detection results against one or more reference materials, adjusting settings (automatically or manually) to cause the detection device's results to match expected or measured results, or performing any other calibration procedures. For example, some embodiments include running one or more calibration modules or functions (e.g., a StereoCameraCalibrate.py function). Some embodiments include calibrating the sensors against a calibration guide 136 having one or more calibration markers 138 (for example, as shown in FIG. 3). Although the calibration guide can include any type of markers (including any of the marker types discussed herein) or any other calibration feature (e.g., lines, reference points, colors, spectra, foci, or other calibration features), some embodiments of the calibration guide include a Charuco board. By way of non-limiting illustration, a specific example of calibration could include one or more of the following: generating a Charuco board (or other calibration guide) specified for calibration; saving the calibration guide (e.g., as {X}×{Y} Charucoboard {length}.png); mounting the calibration guide to a suitable surface (e.g., a planar and rigid surface, or another surface configured to ensure that the calibration markers do not shift with respect to one another); identifying identification points (e.g., corners) of markers; saving the image (such as by pressing a save key (e.g., “S”), selecting an icon, or otherwise presenting a save command) when all identification points are identified (for example, as may be indicated by a red outline or another indication presented by the computer software); capturing a number of images (e.g., between 1 and 10,000, or any subrange thereof, such as between 5-100, between 10-12, or any other suitable number) of the reference guide in different orientations (and in some cases, using the indicated corners or other identification points to make sure a good representation of orientation and position is provided); sending the computer a calibration command (such as by pressing a calibrate button (e.g., “C”), selecting an icon, or otherwise inputting the command); notating any information provided by the computer, such as parameters, error margins (e.g., an RMS reprojection error), or other information; and saving a calibration state (e.g., for individual cameras, for a stereo camera pair, or for any other arrangement of sensors) in any appropriate save format (e.g., a cali.npy file).

With reference now generally to box 240, some embodiments include detecting the markers using the detection device. The markers can be detected with the detection device in any suitable manner, such as through capturing images, capturing sound, detecting radiation, detecting magnetism, or any other detection method, whether intermittently, constantly, in stages, in real time, or otherwise. In some embodiments, detecting the markers is performed as part of running a program, as discussed below, but in some embodiments, it is done prior to (or concurrently with) running the program. In some cases, this involves capturing one or more images of the markers from two or more different positions (where the positions relative to each other are known). In some cases, the images are synchronized, and in some cases, the images are captured using a global shutter.

With reference now to box 250, some embodiments involve running a program for localization of components. In some embodiments, this includes inputting one or more commands to the computer system, such as commands to acquire the position of one or more rigid bodies, measure and compute desired components, clear distances, reposition a robotic component, or otherwise perform any operation useful for a surgical procedure or for localizing a component during any suitable procedure. In some embodiments, the position of at least some of the markers is detected while at least a portion of the instrument is disposed within a patient to perform a surgical procedure. In some embodiments, the 3D position of one or more rigid bodies is calculated based on the sensed position of the markers (e.g., a rigid body of an instrument 110 with a marker guide 130 disposed thereon, as shown in FIG. 1). In some cases, multiple images (or other inputs) are captured, and the movement of markers between images (or other inputs) is used to calculate the movement of the rigid bodies. By way of non-limiting illustration, running the program can include any of the following: running one or more functions (e.g., a main.py function); displaying one or more components (e.g., instruments, implants) on a screen, or otherwise outputting their position to a user; and acquiring a specific position of one or more components (such as by inputting a command (e.g., pressing “A”) to acquire the positions of the implants). As with other portions of the method, some or all of the foregoing can be done repeatedly to acquire as many positions as desired.

Running the program can also include performing additional actions (e.g., through one or more modules or functions). For example, in some cases, the method includes analyzing captured images for individually identifiable fiducial markers. This can include identifying any type of markers, identification points of markers, or other aspects of markers (e.g., if color is used, color can also be used to identify markers). In some cases, the method includes identifying individual markers that appear in multiple images, and selecting those markers for analysis.

According to some embodiments, the method includes determining the coordinates of each marker presented or selected on an image. In some embodiments, captured images are 2-dimensional, so only X and Y image coordinates of the markers are identified on such images, with 3-dimensional coordinates of the associated object being calculated using the 2-dimensional coordinates as reference points. By way of non-limiting illustration, a certain reference point (e.g., an identification point 134 as shown in FIG. 2) may be at (X1, Y1) in a first image and (X2, Y2) in a second image.

According to some embodiments, the method 200 includes combining the marker coordinates. For example, in some cases, the X and Y image coordinates discussed in the previous paragraph are combined between the calibrated images to determine the specific X, Y, and Z positions of the markers represented in the images (e.g., the identification point 134 as shown in FIG. 2 can determined to be at location (X, Y, Z)). Thus, multiple individually identifiable markers may be tracked at once and used together to identify precise (X, Y, Z) locations of markers or identification points. Relatedly, in some embodiments, one or more tables can be generated tracking a large number of marker points or global coordinates, as follows:

Marker Points (Image Coordinates)

| Marker | Image 1 Coordinates | Image 2 Coordinates |
|---|---|---|
| P39_1 | X1, Y1 | X2, Y2 |
| P39_2 | X3, Y3 | X4, Y4 |
| P39_3 | X5, Y5 | X6, Y6 |
| P39_4 | X7, Y7 | X8, Y8 |
| P38_4 | X8, Y8 | X9, Y9 |

Global Coordinates

| Marker | Coordinates |
|---|---|
| P39_1 | X(a), Y(a), Z(a) |
| P39_2 | X(b), Y(b), Z(b) |
| P39_3 | X(c), Y(c), Z(c) |
| P39_4 | X(d), Y(d), Z(d) |
| P38_4 | X(e), Y(e), Z(e) |

According to some embodiments, the method 200 includes calculating an object transformation matrix. While this can be done in any suitable manner, in some embodiments the calculated (X, Y, Z) coordinates of individual markers or a collection of markers are compared to the (X, Y, Z) positions of the corresponding object coordinates to calculate the object transformation matrix. In some cases, calculating the object transformation matrix includes running one or more algorithms. For example, some embodiments involve using Kabsch, Procrustes superimposition work by computing a rotation matrix by reducing the RMS deviation of the collection of points. As another example, some embodiments include running one or more random sample consensus (RANSAC) algorithms (or other algorithms for removing outliers from data) to eliminate noisy data points and reduce error (such as RMS error). In some cases, the method includes dropping individual markers from the combined marker coordinates or from the computation (or part thereof) if the individual markers are not present in one or more images. That said, in some cases, the positions of markers not present in one or more images can be calculated using data from other markers that are present along with the fixed positional association of the markers. As mentioned above, the “positions” calculated and discussed herein can refer to (X, Y, Z) coordinates as well as rotations (RX, RY, RZ). Accordingly, with markers rigidly attached to an object of interest, a rotation matrix can be calculated (which may be in addition to or the same matrix as the matrix for determining (X, Y, Z) coordinates. Thus, in some embodiments, a comprehensive transformation matrix is formed whereby objects can be successfully localized (including coordinates, orientations, and scale) in 3D space, even where substantial portions (or even all) of such objects are not visible (e.g., they are disposed inside a patient or otherwise obscured from view), due to the rigid relationship of detectable markers with such objects. For example, a transformation matrix may include data such as the following (with “T” representing translation, “R” representing rotation, and “S” representing scale):

| SₓR₀₀ | R₀₁ | R₀₂ | Tₓ |
|---|---|---|---|
| R₁₀ | S_{y}R₁₁ | R₁₂ | T_{y} |
| R₂₀ | R₂₁ | S_{z}R₂₂ | T_{z} |
| 0 | 0 | 0 | 1 |

In some embodiments, the method 200 includes calculating one or more variables useful for performing the procedure in question. Such variables can include positions, locations, sizes, distances, orientations, rotations, depths, measurements for additional components, or any other variables that might prove useful during a procedure. For example, in some cases, a desired length for a secondary component 122 may be dependent on the positions of various surgical components 120, and some embodiments of the software are configured to provide recommendations for the desired length based on the positions. By way of non-limiting illustration, FIGS. 4A and 4B show illustrations of digital representations of the positions of implant anchors 120, as calculated by the computer software based on the position of the instrument 110 which is coupled to a marker guide 130 (as shown in FIG. 1) detectable via sensors 152 (as shown in FIG. 5). FIG. 4B shows a secondary component 122 with dimensions (w1 and w2) calculated and recommended via the computer software. Accordingly, by way of further illustration, some embodiments of the method can include any of the following: measuring, computing, or displaying any components (e.g., secondary components that have not yet been inserted into the patient); inputting a command for measurement, computation, or display (e.g., pressing “M” or otherwise inputting the command); clearing (e.g., from short term computer memory) a previously measured distance (such as by inputting a command (e.g., pressing “C”)); or clearing all previously measured distances (such as by inputting a command (e.g., pressing “C” twice).

In some embodiments, the system 100 is used in connection with one or more automated (e.g., robotic) components, such as arms, actuators, robots, or other surgical devices capable of interfacing with the software. Accordingly, in some cases, the automated components are configured to receive input from the software and act accordingly. To illustrate, in some cases, a robotic component is configured to move one or more components 102 to positions calculated using the software. In some cases, this can be done using an input (e.g., pressing “B”) to cause the robot to mimic the calculated locations. In some embodiments, the position of the robot (or one or more components with which the robot is interfacing, such as an instrument 110) is sensed and calculated using the systems and methods discussed herein (for example, some embodiments of the robot include one or more marker guides disposed thereon).

With reference generally to box 260, some embodiments of the method 200 further include physical assembly of components, or locking components into desired positions. In some embodiments, this includes placing surgical components into desired positions (e.g., snapping, locking, hooking, adhering, threadingly coupling, or otherwise coupling components together in the desired configuration) based on the digital representation of the components' localization. In some cases, the components are selected or formed based on measurements or other recommendations provided by the software (e.g., as displayed on the screen 162). In some embodiments, the method 200 includes assembling one or more secondary components based on the position of the components. As discussed above, some embodiments of the method 200 include calculating a desired configuration of the secondary components (e.g., using the software) based on the localization of the components (e.g., and information stored in the memory of the computer system). Accordingly, some embodiments of the method 200 further include assembling the secondary components based on the calculated desired configuration. In some cases, the method 200 further includes locking the components into place using the secondary components. By way of non-limiting illustration, a specific implementation of physical assembly can include any of the following: introducing components into the surgical site; locking components to anchors (e.g., screws), such as anchors previously implanted; using a standard rod locker to lock components into position to make a rod solid; and selecting rods or other components based on length (shorter or longer rods can be used to manipulate the spine in various locations), desired treatment, or other specified parameters. In some embodiments, the method includes running one or more optimization routines to create best-case scenarios for component use.

In some embodiments, the software provides one or more images or animations of any of the portions of the method to be output to the user, such as any of the portions relating to physical assembly of components (e.g., introducing the components into the surgical site, assembly of the implant construct, etc.). In some embodiments, the animations are communicated to the user via the output device 160. In some embodiments, a computer representation of a desired object is displayed on a screen after application of the computed transformation matrix.

In some embodiments, the method 200 includes one or more additional actions as necessary or desirable to allow a practitioner to better localize one or more components or to utilize information stemming from such localization.

### Representative Operating Environment

As mentioned previously, some embodiments of the described system include one or more computers 170. For example, in some embodiments, a device is provided (which may be a handheld device, a mounted device, or another type of device) that includes integrated system components, such as a detection device 150, an output device 160, and a computer. In some cases, the device is configured to be used in connection with a stand 154 (such as a tripod or another type of stand), and in some cases, the device includes a housing 164 holding together the various components and allowing for use as a standalone (e.g., handheld) device. In some embodiments, the device includes input controls 166 (e.g., buttons, switches, touch-screen components, sensors, or other components configured to receive input from a user). By way of non-limiting illustration, FIG. 5 shows a device including a detection device 150 with sensors 152 used in connection with a stand 154 (which can then be used in connection with a mobile device, a computer, a TV, or any other device configured to operate as an output device), and FIGS. 6A-6C show a handheld device that includes a detection device 150 with sensors 152, an output device 160 with a screen 162, a computer 170, a housing 164, and several buttons configured to operate as input controls 166.

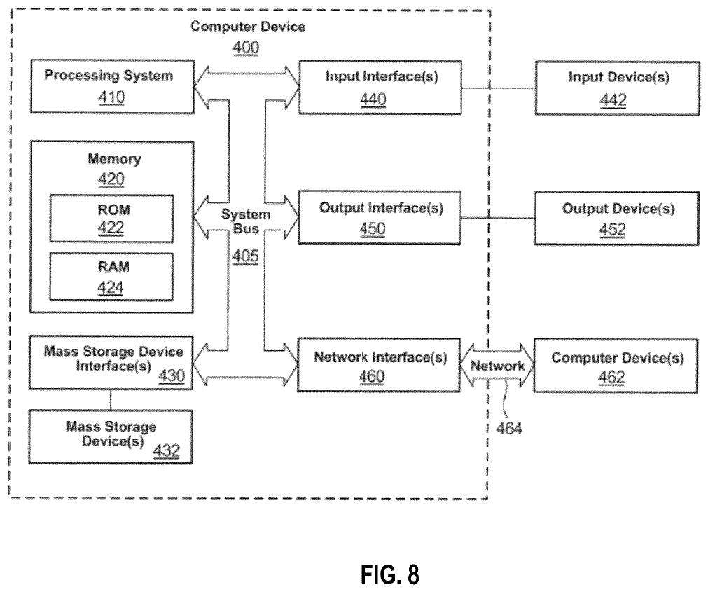

Regarding operation of the computer 170, the described system can be used with, or in, any suitable operating environment and/or software. FIG. 8 and the corresponding discussion are intended to provide a general description of a suitable operating environment in accordance with some embodiments of the described systems and methods. As will be further discussed below, some embodiments embrace the use of one or more processing (including micro-processing) units in a variety of customizable enterprise configurations, including in a networked configuration, which may also include any suitable cloud-based service, such as a platform as a service or software as a service.

Some embodiments of the described systems and methods embrace one or more computer readable media, wherein each medium may be configured to include or includes thereon data or computer executable instructions for manipulating data. In accordance with some embodiments, the computer executable instructions include data structures, objects, programs, routines, or other program modules that can be accessed by one or more processors, such as one associated with a general-purpose processing unit capable of performing various different functions or one associated with a special-purpose processing unit capable of performing a limited number of functions. In this regard, in some embodiments, the processing unit comprises a specialized processing unit (e.g., as shown in FIGS. 6A-6C) that is configured for use with the described system.

Computer executable instructions cause the one or more processors of the enterprise to perform a particular function or group of functions and are examples of program code means for implementing steps for methods of processing. Furthermore, a particular sequence of the executable instructions provides an example of corresponding acts that may be used to implement such steps.

Examples of computer readable media (including non-transitory computer readable media) include random-access memory (“RAM”), read-only memory (“ROM”), programmable read-only memory (“PROM”), erasable programmable read-only memory (“EPROM”), electrically erasable programmable read-only memory (“EEPROM”), compact disk read-only memory (“CD-ROM”), or any other device or component that is capable of providing data or executable instructions that may be accessed by a processing unit.

With reference to FIG. 8, a representative system includes a computer device 400 (e.g., one or more processors, such as the computer 170 discussed above), which may be a general-purpose or special-purpose computer (or processing unit). For example, the computer device 400 may include one or more processors, personal computers, notebook computers, PDAs or other handheld devices, workstations, minicomputers, mainframes, supercomputers, multi-processor systems, network computers, processor-based consumer devices, cellular phones, tablet computers, smart phones, feature phones, smart appliances or devices, control systems, or the like.

The computer device of some embodiments 400 includes a system bus 405, which may be configured to connect various components thereof and enables data to be exchanged between two or more components. The system bus 405 may include one of a variety of bus structures including a memory bus or memory controller, a peripheral bus, or a local bus that uses any of a variety of bus architectures. Typical components connected by the system bus 405 include a processing system 410 and memory 420. Other components may include one or more mass storage device interfaces 430, input interfaces 440, output interfaces 450, or network interfaces 460, each of which will be discussed below.

The processing system 410 of some embodiments includes one or more processors, such as a central processor and optionally one or more other processors designed to perform a particular function or task. It is typically processing system 410 that executes the instructions provided on computer readable media, such as on the memory 420, a magnetic hard disk, a removable magnetic disk, a magnetic cassette, an optical disk, or from a communication connection, which may also be viewed as a computer readable medium.

Memory 420 includes one or more computer readable media (including non-transitory computer readable media) that may be configured to include or includes thereon data or instructions for manipulating data, and may be accessed by processing system 410 through system bus 405. Memory 420 may include, for example, ROM 422, used to permanently store information, or RAM 424, used to temporarily store information. ROM 422 may include a basic input/output system (“BIOS”) having one or more routines that are used to establish communication, such as during start-up of computer device 400. RAM 424 may include one or more program modules, such as one or more operating systems, application programs, and/or program data.

One or more mass storage device interfaces 430 may be used to connect one or more mass storage devices 432 to the system bus 405. The mass storage devices 432 may be incorporated into or may be peripheral to the computer device 400 and allow the computer device 400 to retain large amounts of data. Optionally, one or more of the mass storage devices 432 may be removable from computer device 400. Examples of mass storage devices include hard disk drives, magnetic disk drives, tape drives, solid state mass storage, and optical disk drives.

Examples of solid-state mass storage include flash cards and memory sticks. A mass storage device 432 may read from and write to a magnetic hard disk, a removable magnetic disk, a magnetic cassette, an optical disk, or another computer readable medium. Mass storage devices 432 and their corresponding computer readable media provide nonvolatile storage of data and executable instructions that may include one or more program modules, such as an operating system, one or more application programs, other program modules, or program data. Such executable instructions are examples of program code means for implementing steps for methods disclosed herein.

One or more input interfaces 440 may be employed to enable a user to enter data (e.g., initial information) and instructions to computer device 400 through one or more corresponding input devices 442. Examples of such input devices include a keyboard or alternate input devices, such as one or more switches, buttons, dials, sensors (e.g., temperature sensors, G-force sensors, RPM sensors, color sensors, heart rate sensors, blood pressure sensors, conductivity sensors, sweat sensors, and/or any other suitable type of sensors, including those discussed elsewhere herein), digital cameras (e.g., stereoscopic cameras), stereoscopic sensors, pin pads, touch screens, mice, trackballs, light pens, styluses, or other pointing devices, microphones, joysticks, scanners, camcorders (e.g., stereoscopic camcorders), or other input devices. Similarly, examples of input interfaces 440 that may be used to connect the input devices 442 to the system bus 405 include a serial port, a parallel port, a game port, a universal serial bus (“USB”), a firewire (IEEE 1394), a wireless receiver, a video adapter, an audio adapter, a parallel port, a wireless transmitter, or another interface.

One or more output interfaces 450 may be employed to connect one or more corresponding output devices 452 to system bus 405. Examples of output devices include a monitor or display screen, a speaker, a wireless transmitter, a printer, and the like. A particular output device 452 may be integrated with or peripheral to computer device 400. Examples of output interfaces include a video adapter, an audio adapter, a parallel port, and the like.

One or more network interfaces 460 enable computer device 400 to exchange information with one or more local or remote computer devices, illustrated as computer devices 462, via a network 464 that may include one or more hardwired and/or wireless links. Examples of the network interfaces include a network adapter for connection to a local area network (“LAN”) or a modem, BLUETOOTH™, Wi-Fi, a cellular connection, a wireless link, or another adapter for connection to a wide area network (“WAN”), such as the Internet. The network interface 460 may be incorporated with or be peripheral to computer device 400.

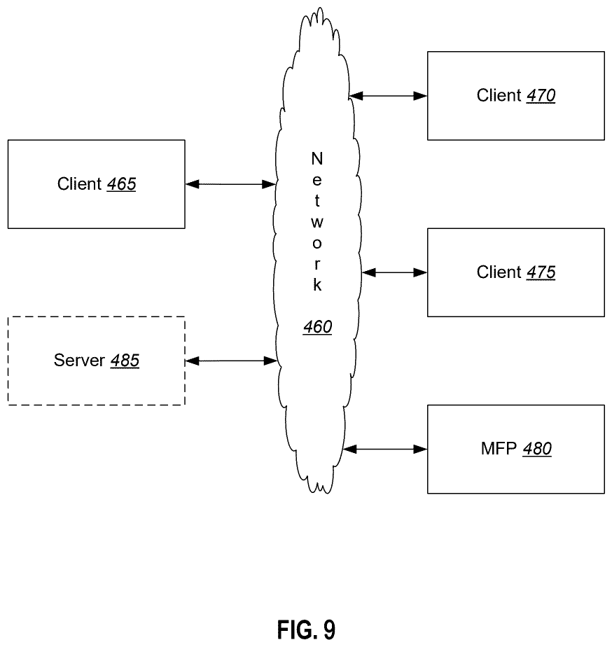

In a networked system, accessible program modules or portions thereof may be stored in a remote memory storage device. Furthermore, in a networked system computer device 400 may participate in a distributed computing environment, where functions or tasks are performed by a plurality networked computer devices. While those skilled in the art will appreciate that the described systems and methods may be practiced in networked computing environments with many types of computer system configurations, FIG. 9 represents an embodiment of a portion of the described systems. While FIG. 9 illustrates an embodiment that includes three clients connected to the network, alternative embodiments include at least one client connected to a network or many clients connected to a network. Moreover, embodiments in accordance with the described systems and methods also include a multitude of clients throughout the world connected to a network, where the network is a wide area network, such as the Internet. Accordingly, in some embodiments, the described systems and methods can allow for remote monitoring, training, communication, observation, control, adjustment, troubleshooting, data collecting, system optimization, user interaction, or other controlling of the described system from one or more places throughout the world.

The systems and methods disclosed herein can be modified in any suitable manner. For example, in some embodiments, the system 100 is capable of internet or other network connectivity. This can allow for real-time guidance during a medical procedure to be offered remotely (e.g., by a skilled professional in another city). It can also allow for parameters or specifications to be communicated to other personnel or locations, such as to a manufacturing facility separate from the operating room in which a procedure is taking place. Thus, a custom part can be fabricated during the procedure to fit with exact measurements of a patient without causing undo delay. The systems and methods can also be used in contexts outside of medical operations, such as automobile repair, manufacturing, product assembly, or any other field that may benefit from positional localization or analysis.

The systems and methods can include any other suitable feature. In this regard, the systems and methods disclosed herein can lead to decreased operating times (thereby reducing the risk of mistake, infection, or other harm), increased accuracy (e.g., better computational solutions leading to more appropriate implant measurement and specifications), greater ease of operation (thereby lowering cost of procedure). The versatility of the systems and methods can also allow for easy setup and transfer to a wide variety of components. Generally speaking, tracking works, in some embodiments, better with larger markers (increased resolution results from increased size). However, larger markers are also, in some embodiments, easier to partially occlude, often making them impractical. By using multiple smaller markers and excluding the occluded ones, it is possible, in some embodiments, to obtain results similar to use of un-occluded large markers, even when some of the smaller markers are occluded. Consequently, the position and orientation of each marker, a rigid body, or an entirely assembly can then be calculated based off the individual marker points combined into one or more arrays. If a marker is not detected, its points can be temporarily removed from the array (or ascribed a lesser weight) and the position and orientation can then be calculated using only the sensed points (or calculated preferentially with a greater weight ascribed to the sensed points).

As the systems and methods disclosed herein are compatible with one another, the systems discussed herein can be used in practicing the methods disclosed herein, and vice versa. Accordingly, the method may further include implementing, exercising, or otherwise using any of the components discussed herein for any of their stated or intended purposes, as reasonably predictable and understood by a person of ordinary skill in the art. The systems disclosed herein can be made in any suitable manner, and they may be used in any way consistent with their operational capabilities. Moreover, in some cases, any particular element or elements of any apparatus—or portion or portions of any method—disclosed herein can be omitted. Additionally, the various components, embodiments, embodiments, cases, elements, figures, and other aspects described herein are not exclusive and can be combined and interchanged in any suitable manner. For instance, any aspect of one embodiment or figure can be combined with any other aspect of one or more embodiments or figures.

As used herein, the singular forms “a”, “an”, “the” and other singular references include plural referents, and plural references include the singular, unless the context clearly dictates otherwise. For example, reference to an instrument includes reference to one or more instruments, and reference to markers includes reference to one or more markers. In addition, where reference is made to a list of elements (e.g., elements a, b, and c), such reference is intended to include any one of the listed elements by itself, any combination of less than all of the listed elements, and/or a combination of all of the listed elements. Moreover, the term “or” by itself is not exclusive (and therefore may be interpreted to mean “and/or”) unless the context clearly dictates otherwise. The term “and” by itself may also be interpreted to mean “and/or” unless the context clearly dictates otherwise. Furthermore, the terms “including”, “having”, “such as”, “for example”, “e.g.”, and any similar terms are not intended to limit the disclosure, and may be interpreted as being followed by the words “without limitation”.

In addition, as the terms “on”, “disposed on”, “attached to”, “connected to”, “coupled to”, etc. are used herein, one object (e.g., a material, element, structure, member, etc.) can be on, disposed on, attached to, connected to, or otherwise coupled to another object-regardless of whether the one object is directly on, attached, connected, or coupled to the other object, or whether there are one or more intervening objects between the one object and the other object. Also, directions (e.g., “front”, “back”, “on top of”, “below”, “above”, “top”, “bottom”, “side”, “up”, “down”, “under”, “over”, “upper”, “lower”, “lateral”, “right-side”, “left-side”, “inside”, “outside”, “base”, etc.), if provided, are relative and provided solely by way of example and for ease of illustration and discussion and not by way of limitation.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, examples, and illustrations set forth herein can be combined in any suitable manner with any other components or characteristics from one or more other embodiments, examples, and illustrations described herein.

What is claimed is:

1. A system for stereotactic localization of an object during a procedure, the system comprising:
a first component;
a first plurality of markers coupled to the first component;
a detection device comprising a sensor configured to detect a position of each of the first plurality of markers; and
an output device,
wherein the system is configured compute a position of the first component, and communicate the position of the first component to a user of the system through the output device.

2. The system of claim 1, wherein the sensor is configured to detect at least two of an x-position, a y-position, a z-position, and a rotation of each of the first plurality of markers.

3. The system of claim 1, wherein the first plurality of markers comprises a first marker that is unique and individually identifiable independently of a second marker.

4. The system of claim 1, wherein each of the first plurality of markers is unique and individually identifiable independently of any other marker.

5. The system of claim 1, wherein each of the first plurality of markers is substantially planar.

6. The system of claim 1, wherein at least two of the first plurality of markers are disposed at different orientations with respect to one another.

7. The system of claim 1, wherein each of the first plurality of markers comprises a plurality of identification points, and wherein the system is configured to determine a marker rotation based on a position of a marker's identification points relative to each other.

8. The system of claim 1, wherein the system comprises an instrument, and wherein the instrument comprises a rigid body.

9. The system of claim 1, wherein the system comprises an instrument, wherein the instrument comprises a shaft and a head, and wherein at least some of the first plurality of markers are disposed on the shaft.

10. The system of claim 9, wherein the instrument is configured to selectively couple to the first component.

11. The system of claim 1, wherein the first component comprises an implant.

12. The system of claim 11, wherein the implant comprises an anchor configured to couple to an implant body.

13. The system of claim 10, wherein the system comprises a memory bank configured to store information relating to the position of the first component after the first component is uncoupled from the instrument, and wherein the system is configured to convey that information to a user through the output device.

14. The system of claim 13, wherein the system is further configured to calculate an optimal configuration for a second component based on the position of the first component.

15. The system of claim 1, further comprising a reference guide configured to be coupled to a patient's body, wherein the system is configured to compute the position of the first component relative to the reference guide.

16. The system of claim 15, wherein the reference guide comprises a reference marker, which is individually identifiable and distinguishable from each of the first plurality of markers.

17. The system of claim 15, wherein the reference guide comprises a plurality of reference markers configured to be coupled to different portions of the patient's body.

18. The system of claim 17, wherein the different portions of the patient's body include different vertebral segments of a spine of the patient.

19. The system of claim 17, wherein the system is further configured to compute a position of the different portions of the patient's body, and to communicate the position of the different portions of the patient's body to the user through the output device.

20. The system of claim 1, wherein the first plurality of markers is substantially disposed in a grid.

21. The system of claim 1, wherein the first plurality of markers comprises at least two markers separated from each other by a gap.

22. The system of claim 1, further comprising:
a second component; and
a second plurality of markers coupled to the second component,
wherein the system is configured compute a position of the second component, and communicate the position of the second component to the user through the output device, and wherein the first component comprises a first bone and the second component comprises a second bone.

23. The system of claim 1, wherein the sensor comprises a first optical sensor and a second optical sensor.

24. The system of claim 1, wherein each of the first plurality of markers comprises an ArUco code.

25. The system of claim 1, wherein the system is calibrated to calculate a transformation matrix even when the sensor cannot detect one or more of the first plurality of markers.

26. A method of stereotactically localizing a component during a surgical procedure, the method comprising:
obtaining a component having a plurality of markers disposed thereon;
obtaining a sensor configured to sense at least one of an x-position, a y-position, a z-position, and a rotation of each of the plurality of markers;
disposing at least a portion of the component within a body of a patient to perform the surgical procedure;
using the sensor to detect at least some of the plurality of markers while the portion of the component is disposed within the body of the patient; and
providing information sensed by the sensor to a processor, the processor being configured to compute a position of the component and communicate the position of the component to a user via an output device.

27. The method of claim 26, wherein the component comprises an instrument.

28. The method of claim 27, wherein the instrument comprises a shaft and a head.

29. The method of claim 28, wherein the head is configured to selectively couple to a surgical component.

30. The method of claim 29, wherein the method further comprises determining a position of the surgical component based on the position of the component as computed by the processor.

31. The method of claim 30, wherein the processor is configured to store in memory the position of the surgical component after the surgical component is uncoupled from the head of the instrument.

32. The method of claim 26, wherein the component comprises a surgical component.

33. The method of claim 31, wherein the surgical component comprises an implant.

34. The method of claim 33, wherein the implant comprises an anchor.

35. A stereotactic localization kit comprising:
a plurality of markers, wherein each of the plurality of markers is individually identifiable and unique from each other of the plurality of markers; and
a device configured to determine a position of each of the plurality of markers and assemble a 3-dimensional digital representation of one or more objects based on the position of each of the plurality of markers.

36. The stereotactic localization kit of claim 35, wherein the device comprises:
a detection device configured to detect the plurality of markers;
a processor configured to determine the position of each of the plurality of markers and assembly the 3-dimensional digital representation of the one or more objects based on the position of each of the plurality of markers; and
an output device configured to convey computed information to a user.

37. The stereotactic localization kit of claim 35, wherein at least some of the plurality of markers are 2-dimensional markers.

38. The stereotactic localization kit of claim 35, wherein the markers are configured to be selectively disposed on a component to be stereotactically located.

39. The stereotactic localization kit of claim 38, wherein the component comprises an instrument.

40. The stereotactic localization kit of claim 38, wherein the component comprises a surgical component.
